(19) United States
(12) Patent Application Publication
(10) Pub. No.: US 2021/0236192 A1
(43) Pub. Date: Aug. 5, 2021

(54) MEDICAL DEVICE SYSTEMS AND METHODS OF USING THE SAME

(71) Applicant: Boston Scientific Scimed Inc., Maple Grove, MN (US)

(72) Inventors: George Wilfred DUVAL, Sudbury, MA (US); Keith R. MAILE, New Brighton, MN (US); James Frederick SAWICKI, Arlington, MA (US); Jan Weber, Maastricht (NL)

(73) Assignee: Boston Scientific Scimed Inc., Maple Grove, MN (US)

(21) Appl. No.: 17/166,904

(22) Filed: Feb. 3, 2021

Related U.S. Application Data
(60) Provisional application No. 62/969,918, filed on Feb. 4, 2020.

(51) Int. Cl. A61B 18/14; A61B 18/18

(52) U.S. Cl. CPC A61B 18/14; A61B 2018/00577; A61B 18/1815

(57) ABSTRACT

A medical instrument that includes a shaft and a device coupled to a distal end of the shaft. The device includes a sensor configured to measure an electrical parameter of tissue at a target site, and an energy delivery device configured to deliver energy to the tissue at the target site based on the measured electrical parameter.

### CROSS-REFERENCE TO RELATED APPLICATIONS

This application claims the benefit of priority from U.S. Provisional Application No. 62/969,918, filed Feb. 4, 2020, which is incorporated by reference herein in its entirety.

### TECHNICAL FIELD

Various aspects of the disclosure relate generally to medical diagnostic and therapy instruments, systems, devices, and related methods. More specifically, examples of the disclosure relate to instruments, systems, devices, and related methods for conducting diagnostic tests to locate one or more target sites within a patient during an endoscopic procedure and applying energy therapy thereto, among other aspects.

### BACKGROUND

Technological developments have given users of medical systems, devices, and methods, the ability to conduct increasingly complex procedures on subjects. One challenge in the field of minimally invasive surgeries such as endoscopy, among other surgical procedures, is associated with the assessment of tissue heath and identification of undesirable tissue (e.g., at-risk, unhealthy, pre-cancerous, cancerous, etc.) within a patient by observing an exterior surface of the tissue. Optical examination of an exterior surface of tissue may provide limited diagnostic analysis of the tissue health as an outer appearance of said surface may not be accurately indicative of actual tissue conditions. The limitations of medical devices in providing diagnostic analysis at various depths of the tissue beyond an exterior surface may prolong the procedure, limit its effectiveness, and/or cause injury to the patient due to misdiagnosis of the tissue.

### SUMMARY

Aspects of the disclosure relate to, among other things, systems, devices, instruments, and methods for locating target treatment sites based on sensing electrical parameters that are indicative of tissue health and treating undesirable tissue with said medical instrument, among other aspects. Each of the aspects disclosed herein may include one or more of the features described in connection with any of the other disclosed aspects.

According to an example, a medical instrument may include a shaft and a device coupled to a distal end of the shaft. The device includes a sensor configured to measure an electrical parameter of tissue at a target site, and an energy delivery device configured to deliver energy to the tissue at the target site based on the measured electrical parameter.

Any of the medical instruments described herein may include any of the following features. The sensor and the energy delivery device are movable relative to the distal end of the shaft. The device includes an expandable body that is configured to expand laterally outward from a collapsed state to an expanded state. The sensor and the energy delivery device are positioned on the expandable body. The sensor includes one or more microwave antennas configured to sense dielectric permittivity of the tissue at the target site. The sensor includes one or more microwave or RF biosensors configured to sense parameters of the tissue at the target site. The sensor includes at least two electrodes configured to measure impedance between the at least two electrodes when positioned adjacent to the target site. The energy delivery device includes a microwave or RF electrode configured to transmit electric current to the target site and generate heat to ablate the tissue at the target site. The medical instrument includes a processor and non-transitory computer readable medium storing instructions that, when executed by the processor, causes the processor to activate the energy delivery device when the electrical parameter measured by the sensor is indicative of undesirable tissue at the target site. The instructions stored in the non-transitory computer readable medium causes the processor to determine whether the electrical parameter measured by the sensor is indicative of undesirable tissue at the target site. The instructions further cause the processor to transmit electrical energy from a generator to the energy delivery device in response to determining the electrical parameter is indicative of undesirable tissue. The instructions stored in the non-transitory computer readable medium cause the processor to periodically re-measuring the electrical parameter at the target site with the sensor after delivering electrical energy from the energy deliver device to the tissue at the target site. The instructions stored in the non-transitory computer readable medium cause the processor to cease delivering electrical energy from the energy delivery device to the tissue at the target site when the electrical parameter measured by the sensor is not indicative of undesirable tissue at the target site. The instructions stored in the non-transitory computer readable medium cause the processor to cease transmission of electrical energy from the generator to the energy delivery device when the electrical parameter measured by the sensor is not indicative of undesirable tissue at the target site. The medical instrument includes a plurality of sensors disposed along an exterior of the device in a first array, and a plurality of energy delivery devices disposed along the exterior of the device in a second array. The device is configured to locate the undesirable tissue relative to the exterior of the device based on a spatial distribution of the first array of the plurality of sensors.

According to another example, a medical instrument may include a flexible shaft having an articulable distal end and a device coupled to the articulable distal end of the shaft. The device including an expandable body, a sensor array disposed along the expandable body and configured to measure an electrical parameter of tissue at a target site, and an electrode array disposed along the expandable body and configured to heat the tissue at the target site. The expandable body is expandable to position the sensor array and the electrode array adjacent to the tissue at the target site.

Any of the medical instruments described herein may include any of the following features. The sensor array includes a plurality of biosensors and the electrode array includes a plurality of electrodes. The plurality of biosensors are disposed along the expandable body and alternate with the plurality of electrodes. The medical instrument may include a processor and non-transitory computer readable medium storing instructions that, when executed by the processor, causes the processor to determine whether the electrical parameter measured by the sensor array is indicative of undesirable tissue at the target site. The instructions cause the processor to activate the electrode array when the electrical parameter measured by the sensor array is indicative of undesirable tissue at the target site. The instructions cause the processor to periodically re-measure the electrical parameter at the target site with the sensor array after delivering electrical energy from the electrode array to the tissue at the target site. The instructions cause the processor to cease delivering electrical energy from the electrode array to the tissue at the target site when the electrical parameter measured by the sensor is not indicative of undesirable tissue at the target site.

According to an exemplary method of treating a target site with a medical device, the method may include (a) measuring an electrical parameter of tissue at the target site with a sensor of the medical device, (b) determining whether the measured electrical parameter is indicative of undesirable tissue, and (c) actuating an energy delivery device of the medical device if the electrical parameter is indicative of undesirable tissue, wherein actuation of the energy delivery device ablates the undesirable tissue. The method may include repeating steps (a) through (c) until the electrical parameter measured at step (a) is determined to not be indicative of undesirable tissue at step (b).

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a schematic view of an exemplary medical system, according to aspects of this disclosure;

FIG. 2 is a partial perspective view of a medical device of the medical system of FIG. 1, according to aspects of this disclosure;

FIG. 3A is a schematic view of the medical system of FIG. 1 positioned at a target site of a patient, according to aspects of this disclosure;

FIG. 3B is a schematic view of the medical system of FIG. 1 at a target site of a patient with the medical device in a deployed state, according to aspects of this disclosure;

FIG. 3C is a schematic view of the medical system of FIG. 1 at a target site of a patient with the medical device in an actuated state, according to aspects of this disclosure;

FIG. 4 is a block diagram of an exemplary method of locating and treating a target site with the medical system of FIG. 1, according to aspects of this disclosure;

FIG. 5 is a partial perspective view of an exemplary medical device, according to aspects of this disclosure; and

FIG. 6 is a partial perspective view of another exemplary medical device, according to aspects of this disclosure.

### DETAILED DESCRIPTION

Examples of the disclosure include systems, devices, and methods for sensing, locating, and/or treating one or more target treatment sites within a subject (e.g., patient) that include undesirable tissue (e.g., unhealthy tissue, pre-cancerous or cancerous cells, tumors, at-risk material, etc.). Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may be used to locate a target site with a medical instrument, such as, for example, a medical instrument having diagnostic sensing logic. Examples of the disclosure may be further used to treat a target site with a medical instrument, such as, for example, a medical instrument having therapy progress logic. For example, some embodiments may combine a medical instrument with a diagnostic computing device to locate and treat a target site. The diagnostic computing device may execute one or more logic operations of the medical instrument at a target site to apply diagnostic and treatment operations at the target site in response to the diagnostic computing device identifying a location of the target site that includes undesirable tissue. The therapy progress logic of the medical instrument may monitor a progress of the treatment operation to detect and/or measure real-time conditions of the tissue at the target site to determine whether the undesirable tissue has been adequately and/or fully treated.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Reference will now be made in detail to examples of the disclosure described above and illustrated in the accompanying drawings. Wherever possible, the same reference numbers will be used throughout the drawings to refer to the same or like parts.

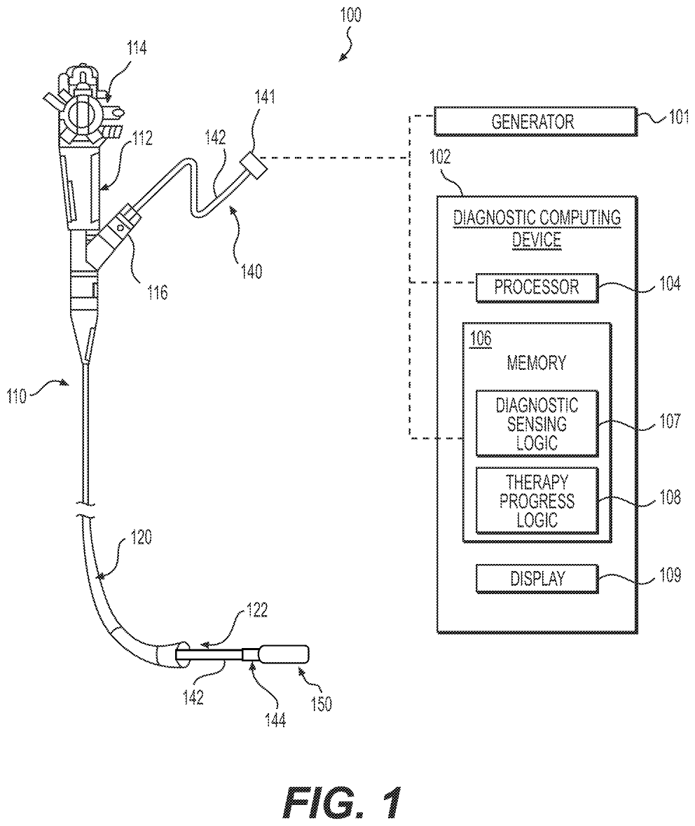

FIG. 1 shows a schematic depiction of an exemplary medical system 100 in accordance with an example of this disclosure. The medical system 100 may include a generator 101, a diagnostic computing device 102, a medical device 110, and a medical instrument 140. The diagnostic computing device 102 may be communicatively coupled to the medical instrument 140 by, for example, a wired connection, a wireless connection, and the like. In examples, the diagnostic computing device 102 is a computer system incorporating a plurality of hardware components that allow the diagnostic computing device 102 to receive and monitor data (e.g., electrical parameters of tissue), initiate delivery of electrical energy (e.g., from the generator 101), and/or process other information described herein. Illustrative hardware components of the diagnostic computing device 102 may include at least one processor 104, at least one memory 106, and at least one display 109.

The processor 104 of the diagnostic computing device 102 may include any computing device capable of executing machine-readable instructions, which may be stored on a non-transitory computer-readable medium, such as, for example, the memory 106 of the diagnostic computing device 102. By way of example, the processor 104 may include a controller, an integrated circuit, a microchip, a computer, and/or any other computer processing unit operable to perform calculations and logic operations required to execute a program. As described in greater detail herein, the processor 104 is configured to perform one or more operations in accordance with the instructions stored on the memory 106, such as, for example, a diagnostic sensing logic 107, a therapy progress logic 108, and the like.

Still referring to FIG. 1, the memory 106 of the diagnostic computing device 102 includes a non-transitory computer readable medium that stores machine-readable instructions thereon, such as, for example, the diagnostic sensing logic 107 and the therapy progress logic 108. As described in further detail below, the diagnostic sensing logic 107 may include executable instructions that allow the medical instrument 140 to detect and/or measure electrical parameters of tissue at a target site to determine whether the target site includes undesirable tissue (e.g., unhealthy tissue, pre-cancerous or cancerous cells, tumors, at-risk material, etc.) that requires treatment. Further, the therapy progress logic 108 may include executable instructions that allow the medical instrument 140 to initiate treatment of the target site by applying energy therapy thereto in response to measuring electrical parameters that are indicative of undesirable tissue at the target site. The therapy progress logic 108 may further include executable instructions that allow the medical instrument 140 to periodically check a progress of the treatment at the target site by ceasing application and/or delivery of the energy therapy and re-measuring electrical parameters of the tissue at the target site, in accordance with the diagnostic sensing logic 107 described above.

It should be appreciated that the therapy progress logic 108 may execute the periodic assessments described herein automatically without requiring user input. In other embodiments, the diagnostic computing device 102 may be configured to receive user inputs to initiate assessment of the treatment progress at the target site, such as, for example, from a user input in communication (e.g., wireless, wired, etc.) with the diagnostic computing device 102.

It should be understood that various programming algorithms and data that support an operation of the medical instrument 140 of the medical system 100 may reside in whole or in part in the memory 106. The memory 106 may include any type of computer readable medium suitable for storing data and algorithms, such as, for example, random access memory (RAM), read only memory (ROM), a flash memory, a hard drive, and/or any device capable of storing machine-readable instructions. The memory 106 may include one or more data sets, including, but not limited to, diagnostic data from one or more components of the medical system 100 (e.g., the medical device 110, the medical instrument 140, etc.).

Still referring to FIG. 1, the medical device 110 may be configured to facilitate positioning one or more components of the medical system 100 relative to a subject (e.g., a patient), such as, for example, the medical instrument 140. In embodiments, the medical device 110 may be any type of endoscope, duodenoscope, gastroscope, colonoscope, ureteroscope, bronchoscope, catheter, or other delivery system, and may include a handle 112, an actuation mechanism 114, at least one port 116, and a shaft 120. The handle 112 of the medical device 110 may have one or more lumens (not shown) that communicate with a lumen(s) of one or more other components of the medical system 100. The handle 112 further includes the at least one port 116 that opens into the one or more lumens of the handle 112. As described in further detail herein, the at least one port 116 is sized and shaped to receive one or more instruments therethrough, such as, for example, the medical instrument 140 of the medical system 100.

The shaft 120 of the medical device 110 may include a tube that is sufficiently flexible such that the shaft 120 is configured to selectively bend, rotate, and/or twist when being inserted into and/or through a subject's tortuous anatomy to a target treatment site. The shaft 120 may have one or more lumens (not shown) extending therethrough that include, for example, a working lumen for receiving instruments (e.g., the medical instrument 140). In other examples, the shaft 120 may include additional lumens such as a control wire lumen for receiving one or more control wires for actuating one or more distal parts/tools (e.g., an articulation joint, an elevator, etc.), a fluid lumen for delivering a fluid, an illumination lumen for receiving at least a portion of an illumination assembly (not shown), and/or an imaging lumen for receiving at least a portion of an imaging assembly (not shown).

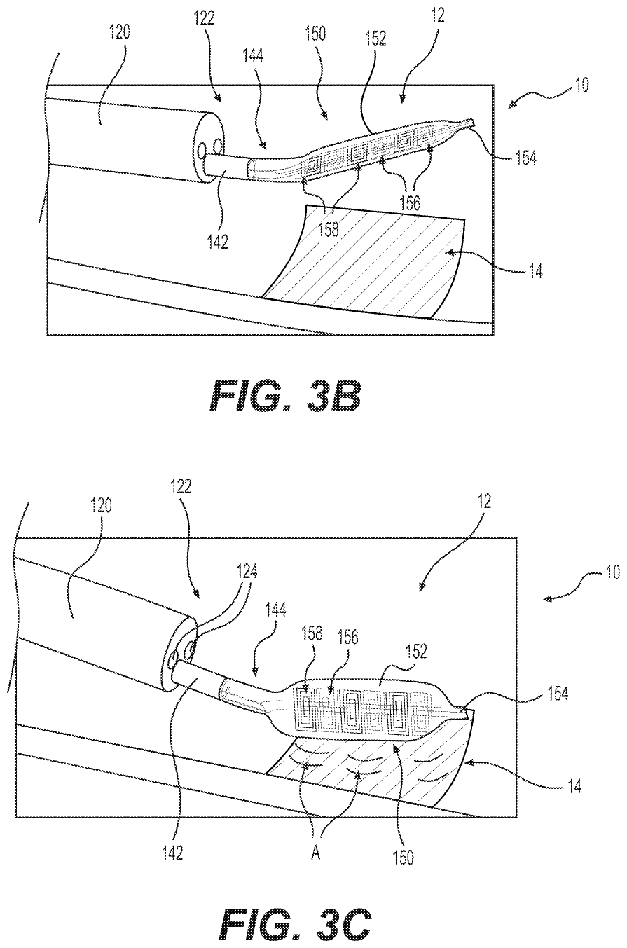

Still referring to FIG. 1, the medical device 110 may further include a tip 122 at a distal end of the shaft 120. In some embodiments, the tip 122 may be attached to the distal end of the shaft 120, while in other embodiments the tip 122 may be integral with the shaft 120. For example, the tip 122 may include a cap configured to receive the distal end of the shaft 120 therein. The tip 122 may include one or more openings 124 that are in communication with the one or more lumens of the shaft 120 (FIGS. 3A-3C). For example, the tip 122 may include a working opening 124A through which the medical instrument 140 may exit from a working lumen of the shaft 120.

In other examples, the tip 122 of the shaft 120 may include additional and/or fewer openings 124 thereon, such as, for example, a fluid opening or nozzle through which fluid may be emitted from a fluid lumen of the shaft 120, an illumination opening/window through which light may be emitted, and/or an imaging opening/window for receiving light used by an imaging device to generate an image. The actuation mechanism 114 of the medical device 110 is positioned on the handle 112 and may include one or more knobs, buttons, levers, switches, and/or other suitable actuators. The actuation mechanism 114 is configured to control at least one of deflection of the shaft 120 (e.g., through actuation of a control wire), delivery of a fluid, emission of illumination, and/or various imaging functions.

Still referring to FIG. 1, the medical instrument 140 of the medical system 100 may include a catheter having a longitudinal body 142 between a proximal end of the medical instrument 140 and a distal end of the medical instrument 140. The longitudinal body 142 of the medical instrument 140 is flexible such that the medical instrument 140 is configured to bend, rotate, and/or twist when being inserted into a working lumen of the medical device 110. A handle 141 is at the proximal end of the longitudinal body 142 and an articulation joint 144 is at the distal end of the longitudinal body 142. The handle 141 of the medical instrument 140 may be configured to move, rotate, and bend the longitudinal body 142. Further, the handle 141 may define one or more ports (not shown) sized to receive one or more tools through the longitudinal body 142 of the medical instrument 140.

The medical device 110 is configured to receive the medical instrument 140 via the at least one port 116, through the shaft 120 via a working lumen, and to the working opening 124A at the tip 122. In this instance, the medical instrument 140 may extend distally out of the working opening 124A and into a surrounding environment of the tip 122, such as, for example, at a target treatment site of a subject as described in further detail below. The distal end of the medical instrument 140, including the articulation joint 144, may extend distally from the working opening 124A in response to a translation of the longitudinal body 142 through the working lumen of the shaft 120. Additionally, the medical instrument 140 may include a combined device 150 at the distal end of the longitudinal body 142. In the example, the combined device 150 includes a probe that is positioned and/or extends distally relative to the articulation joint 144 of the medical instrument 140. The articulation joint 144 of the medical instrument 140 is configured to articulate the combined device 150 relative to a longitudinal axis of the longitudinal body 142. In other words, the articulation joint 144 is operable to bend, deflect, and pivot, the combined device 150 relative to a distal end of the longitudinal body 142 in response to articulation of the articulation joint 144.

As described in further detail herein, the combined device 150 of the medical instrument 140 may include one or more components for diagnosing and treating undesirable tissue at a target treatment site. It should be understood that, in other examples, the medical device 110 and/or the medical instrument 140 may include various other devices than those show and described herein, including but not limited to, a guidewire, cutting or grasping forceps, a biopsy device, a snare loop, an injection needle, a cutting blade, scissors, a retractable basket, a retrieval device, an ablation and/or electrophysiology catheter, a stent placement device, a surgical stapling device, a balloon catheter, a laser-emitting device, an imaging device, and/or any other suitable instrument.

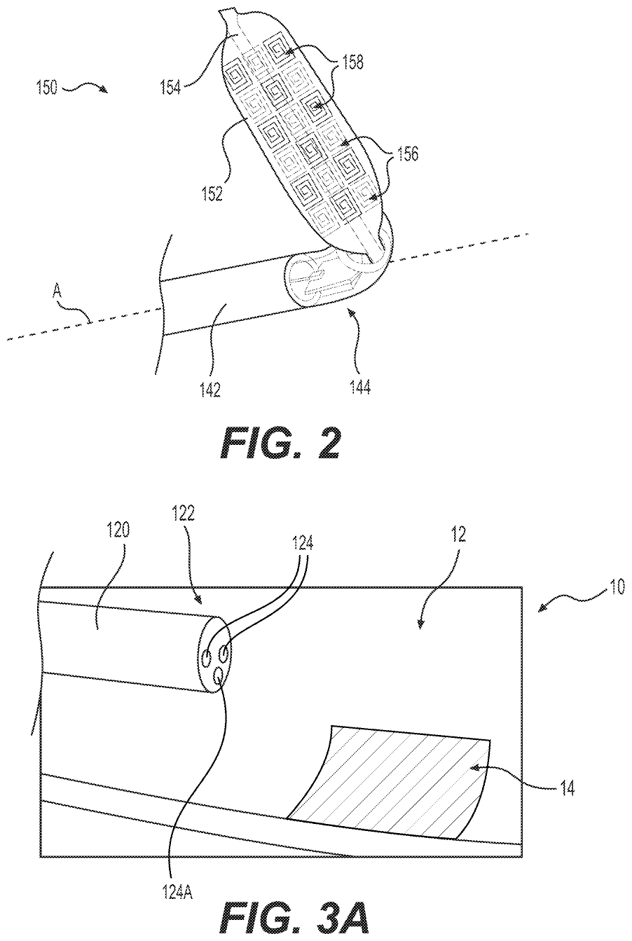

Referring now to FIG. 2, a distal end of the longitudinal body 142 is depicted with the articulation joint 144 of the medical instrument 140 in an articulated state, thereby deflecting the combined device 150 relative to an axis A defined by a distal end of the longitudinal body 142. In the example of FIG. 2, the articulation joint 144 is depicted in an articulated state such that the combined device 150 is positioned at a transverse orientation and/or configuration relative to the axis A of the longitudinal body 142. It should be appreciated that the articulation joint 144 may be configured and operable to articulate (e.g., bend, deflect, pivot, etc.) the combined device 150 to a plurality of orientations and/or configurations relative to the axis A of the longitudinal body 142. As described further below, the articulation joint 144 is configured to position the combined device 150 proximate to a target treatment site during use of the medical system 100 in a procedure. The articulation joint 144 may include any suitable structure, including, for example, a flexible tube, discrete stacked rings that may pivot relative to one another via one or more actuation wires, and the like.

The combined device 150 may include an expandable body 152 having a longitudinal length defined by a proximal end of the combined device 150, positioned adjacent to the articulation joint 144, and a distal end of the combined device 150 (positioned at a terminal end of the expandable body 150, opposite of the proximal end. The combined device 150 may be substantially cylindrical along a longitudinal axis and be formed of a non-compliant material. The combined device 150 may further include an internal rod 154 disposed within the expandable body 152 and having a longitudinal length substantially similar to a longitudinal length of the expandable body 152. Although not shown, it should be appreciated that the internal rod 154 of the combined device 150 may include one or more apertures disposed thereon for transmitting a fluid (e.g., pressurized gas, air, liquid, etc.) within the expandable body 152.

In an example, the internal rod 154 is configured to transition the expandable body 152 from a compressed, compact state (FIG. 3B) to an expanded state (FIG. 3C). In other words, the internal rod 154 is operable to expand and/or compress a size, shape, configuration, and/or profile of the expandable body 152 in response to a transmission or extraction of fluid within the expandable body 152 via the one or more apertures. In other examples, the internal rod 154 is operable to unfold and/or fold the expandable body 152 between the compact state (FIG. 3B) and the expanded state (FIG. 3C). It should be appreciated that the expandable body 154 is configured to transition between the configurations shown and described herein at least in part due to being formed of the non-compliant material.

Still referring to FIG. 2, in some examples, the expandable body 152 of the combined device 150 may include an inflatable balloon, a mesh, a cage, a probe, a stent, a coil, and/or various other suitable devices capable of selective lateral expansion from a compressed state to an expanded state. The combined device 150 may further include at least one diagnostic sensing device 156 and at least one energy delivery device 158. In the example, the combined device 150 may include a plurality of diagnostic sensing devices 156 disposed in an annular array about a circumference of the expandable body 152. In some examples, the diagnostic sensing devices 156 may be disposed within the expandable body 152 of the combined device 150. Further, the combined device 150 may include a plurality of energy delivery devices 158 disposed in an annular array about a circumference of the expandable body 152. The diagnostic sensing devices 156 and the energy delivery devices 158 may face radially outward from a central longitudinal axis (not shown) of the expandable body 152.

The diagnostic sensing devices 156 and the energy delivery devices 158 may be received within apertures (not shown) formed along the expandable body 152 of the combined device 150. In some examples, the apertures of the expandable body 152 may include a split ring resonator (e.g., a resonant circuit) that may form a ground plane, where the split ring resonator may be configured and operable to perform resonate sensing. In this instance, a size of the split ring resonator may vary in correspondence to a material composition of the substrate received within the aperture. For example, a size of the split ring resonator and the aperture formed along the expandable body 152 of the combined device 150 may be minimized when the substrate received therein is formed of a high permittivity dielectric material (e.g., a flexible PCB material). It should be understood that, in other examples, inclusion of a substrate and/or material with the split ring resonator in the aperture may be omitted without departing from a scope of this disclosure. In some examples, the split ring resonator may be sized approximately 1 millimeter squared. In other examples, a spiral resonator may be disposed within the aperture in lieu of the split ring resonator.

As seen in FIG. 2, the array of the diagnostic sensing devices 156 are positioned along the expandable body 152 with the array of the energy delivery devices 158 in a predefined pattern. The predefined pattern of the array of diagnostic sensing devices 156 and the energy delivery devices 158 may be any pattern. Devices 156, 158 may be uniformly distributed about a circumference of the expandable body 152, or may be concentrated in a selected portion of the expandable body 152. In the example of FIG. 2, the combined device 150 includes about three annular arrays of the diagnostic sensing devices 156 and about three annular arrays of the energy delivery devices 158, with the arrays of the diagnostic sensing devices 156 alternating with the arrays of the energy delivery devices 158 along the expandable body 152. It should be understood, however, that the diagnostic sensing devices 156 and the energy delivery devices 158 of the combined device 150 may be positioned along the exterior surface of the expandable body 152 in various other suitable patterns and/or respective locations relative to one another.

Each of the plurality of diagnostic sensing devices 156 is configured to detect and/or measure electrical parameters of a biological material at a target treatment site, such as, for example, a tissue. As described in further detail herein, the electrical parameters measured by the plurality of diagnostic sensing devices 156 (e.g., tissue permittivity, conductivity, impedance, etc.) may be utilized to determine one or more characteristics and/or properties of the tissue at the target treatment site, such as, for example, characteristics indicative of a tissue health and/or condition at the target treatment site. It should be appreciated that the diagnostic sensing devices 156 of the combined device 150 may be operable to provide diagnostic analysis of the target treatment site beyond a surface layer of the tissue (e.g., within a mucosa layer). In other words, with the diagnostic sensing devices 156 configured to sense a presence of electrical parameters of the tissue, the combined device 150 may be capable of conducting a diagnostic of the target site at varying tissue depths that exceeds an assessment of the tissue at the surface layer.

It should be understood that desirable tissue (e.g., tissue including healthy, natural, and/or normal biomolecules or cells) and undesirable tissue (e.g., tissue including unhealthy, unnatural, and/or abnormal molecules or cells) may include relatively varying electrical properties. By way of illustrative example only, undesirable tissue may include harmful properties, pre-cancerous cells, cancerous cells, at-risk material, tumors, and the like. Tissues may include varying impedances, as defined by permittivity and conductivity, depending on an excitation frequency during a diagnostic sensing or therapy procedure of the tissue at the target site by the medical device 100. Examples of the dielectric property differences between undesirable and desirable tissues may include undesirable tissues having different (e.g., relatively higher) permittivity and conductivity than desirable tissues along various microwave frequencies. For instance, the different permittivity and/or conductivity of undesirable tissues compared to desirable tissues may be due to variances in a water and/or chemical composition of the tissue.

In some examples, the diagnostic sensing devices 156 may be a passive element (e.g., a two-terminal device with no power applied) or an active element (e.g., a powered sensor integrated circuit) configured to take measurements. It should be appreciated that when the diagnostic sensing devices 156 include a passive element, power and/or electric current may be supplied to the diagnostic sensing devices 156 by one or more other components of the medical device 100. By way of example, the diagnostic sensing device 156 may include a sensor, including, but not limited to, a microwave biosensor, an RF biosensor, a microwave antenna, and the like. In this instance, the diagnostic sensing device 156 may be configured to detect and/or measure dielectric permittivity of the tissue at the target treatment site, measure an electrical impedance between adjacent diagnostic sensing devices 156, measure an electric charge emitted by the tissue, measure a chemical substance in the tissue, measure a current induced in the tissue, and/or the like.

In some examples, the diagnostic sensing devices 156 may further include a transducer (not shown) that is operable to convert the measured electric energy into a signal (e.g., analog) for transmission to the diagnostic computing device 102. As described in detail above, the data transmitted to the diagnostic computing device 102 by the diagnostic sensing devices 156 (e.g., via analog signal from a transducer of the diagnostic sensing device 156) may be utilized to determine characteristics and/or properties of the tissue at the target treatment site, such as those indicative of a tissue health at the target treatment site. The diagnostic sensing devices 156 and the energy delivery devices 158 may be connected in series with one another and the generator 101. In other examples, the diagnostic sensing devices 156, the energy delivery devices 158, and/or the generator 101 may be electrically connected via various other suitable configurations, such as, for example, a parallel connection.

Still referring to FIG. 2, each of the plurality of energy delivery devices 158 is configured to deliver energy to a biological material at a target treatment site, such as, for example, a tissue. As described in further detail herein, the energy delivered by the plurality of energy delivery devices 158 may be in response to the electrical parameters measured by the plurality of diagnostic sensing devices 156, such as, for example, when the electrical parameters are determined to indicate a target treatment site including undesirable tissue. It should be understood that the energy delivery devices 158 of the combined device 150 may be operable to transmit energy current (e.g., RF current) to the target treatment site to remove or otherwise kill the undesirable tissue by applying resistive heating thereto.

In other words, and as described further herein, the energy delivery devices 158 may provide for a surgical removal (ablation) of the tissue from the target treatment site by generating and transmitting heat via electric current to the undesirable tissue 14. In some instances, an energy level delivered to the tissue from the energy delivery devices 158 may correspond to various factors, including, for example, a predetermined or selected depth of tissue ablation at the target site, a predetermined or selected area of undesirable tissue measured at the target site, and the like. In some examples, the energy delivery devices 158 may include an electrode, such as, for example, a microwave or RF electrode, a bipolar electrode, a monopolar electrode, and the like, or combinations thereof.

Referring back to FIG. 1, with the medical instrument 140 electrically coupled to the generator 101 of the medical system 100 (FIG. 1), the energy and/or electrical current transmitted by the electrodes of the energy delivery devices 158 to the target treatment site may be received from and generated by the generator 101. The generator 101 may include an electrical surgical (i.e., electrosurgical) generator configured to produce a variety of electrical waveforms, including, for example, energy currents (e.g., RF currents) ranging from approximately 100 kilohertz (KHz) to 500 kilohertz (KHz), such as 400 kilohertz (KHz); or microwave frequencies ranging from approximately 1 gigahertz (GHz) to 10 gigahertz (GHz). It should be understood that the generator 101 may be a general-purpose electrosurgical generator that may be utilized with a variety of applications and/or devices, and is not limited to use with the medical instrument 140.

The generator 101 may be electrically and/or physically connected to the energy delivery devices 158 of the combined device 150 via one or more connections (not shown), including, for example, one or more supply lines. The generator 101 is configured to supply the combined device 150 of the medical instrument 140 with electrical energy (e.g., RF current) for delivery from the energy delivery devices 158 to, for example, tissue at a target treatment site. In other examples, the medical system 100 may include various other suitable energy sources in lieu of the generator 101 shown and described above without departing from a scope of this disclosure.

Still referring to FIG. 1, the diagnostic computing device 102 may further include a display 109 that is operable to output information regarding measurements and/or calculations obtained by and/or derived from the combined device 150. For example, the display 109 of the diagnostic computing device 102 may visually and/or audibly display information regarding a status of the combined device 150, sensor data measured by the diagnostic sensing devices 156, a progress of ablation by the energy delivery devices 158, and the like. The display 109 may be configured to output such information in any suitable format, including, for example, a table, a graph, a graphical representation, a list, a present value, a color coding, a combination thereof, and the like. For example, the display 109 may display a graph of frequency measured versus conductivity and/or relative permittivity in accordance with dielectric properties of a tissue at the target treatment site.

The display 109 may further display information regarding a depth of tissue ablation, a progress of tissue ablation, and the like. By way of illustrative example, the display 109 may use a color-coded system such that predefined colors may provide information regarding a progress or status of the ablation by the combined device 150 relative to amount of remaining undesirable tissue at the target site. In this instance, green may indicate a requirement to continue ablation, yellow may indicate that ablation is nearing completion, and red may indicate conclusion of ablation by the combined device 150. In some examples, the display 109 may be configured to interact with and/or receive inputs from a user of the diagnostic computing system 102 to control one or more components of the medical system 100 (e.g., the medical instrument 140) and/or to customize information displayed on the display 109 during a procedure.

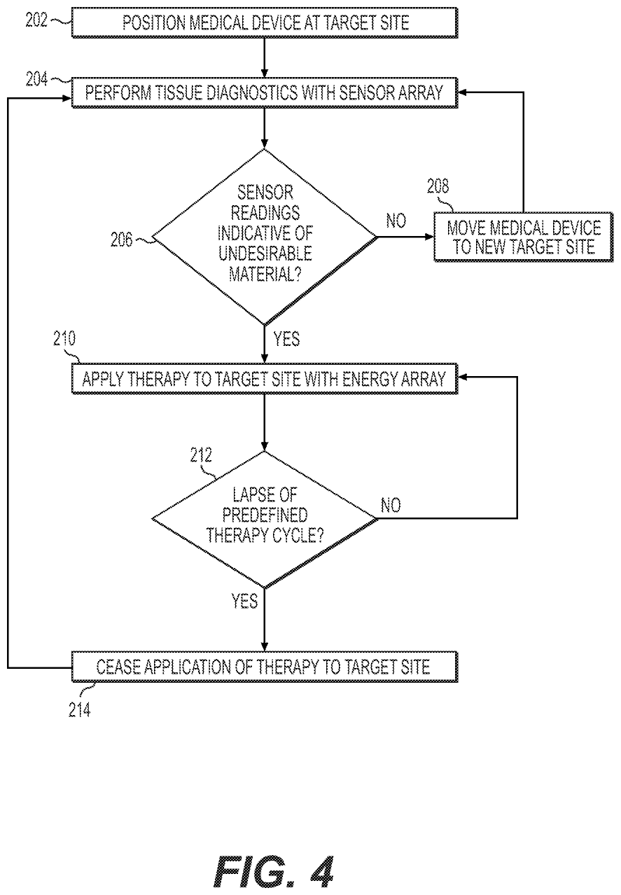

Referring now to FIGS. 3A-3C in conjunction with the flow diagram of FIG. 4, an exemplary method 200 of using the medical system 100 to locate and treat a target site is schematically depicted. The depiction of FIGS. 3-4 and the accompanying description below is not meant to limit the subject matter described herein to a particular method. At step 202, the medical device 110 of the medical system 100 may be inserted within a subject's body (not shown) to position the distal end 122 adjacent to a target site 10. For example, the shaft 120 may be guided through a digestive tract of the subject by inserting the tip 122 into a nose or mouth (or other suitable natural body orifice) of the subject's body and traversed through a gastrointestinal tract of the subject's body (e.g., an esophagus, a stomach, a small intestine, etc.) until reaching the target site 10. It should be appreciated that a length of the shaft 120 may be sufficient so that a proximal end of the medical device 110 (including the handle 112) is external of the subject while the tip 122 of the medical device 110 is internal to the subject's body. While this disclosure relates to the use of the medical system 100 in a digestive tract of a subject, it should be understood that the features of this disclosure could be used in various other locations (e.g., other organs, tissue, etc.) within a subject's body.

As shown in FIG. 3A, with the medical device 110 received within the subject's body, the distal end 122 of the shaft 120 may be located relatively adjacent to the target site 10. The target site 10 may include, inter alia, one or more of desirable tissues 12 and/or undesirable tissues 14. In this instance, the medical instrument 140 may be received within the medical device 110 via the at least one port 116 such that the longitudinal body 142 of the medical instrument 140 is translated through the shaft 120 of the medical device 110 via at least one of the lumens of the shaft 120 (e.g., a working lumen). A distal end of the longitudinal body 142 may be extended distally from the distal end 122 of the shaft 120 via the one or more openings 124, such as, for example, the working opening 124A which is in communication with a working lumen of the shaft 120.

Referring now to FIG. 3B, at least a distal portion of the medical instrument 140 extending distally from the distal end 122 of the shaft 120, including the distal end of the longitudinal body 142 and the combined device 150, a user may articulate the articulation joint 144 to adjust a position, location, and/or orientation of the combined device 150 relative to the target site 10. For example, the articulation joint 144 may be articulated in response to actuating the handle 141 of the medical instrument 140 at a proximal end of the longitudinal body 142. In this instance, it should be appreciated that the expandable body 152 of the combined device 150 is in a compressed, compact state. With the combined device 150 positioned outward from a working lumen of the shaft 120 of the medical device 110, a user may actuate the expandable body 152 to transition the combined device 150 from the compressed state to an expanded state, as seen in FIG. 3C.

Referring to FIG. 3C, with the expandable body 152 expanded and positioned adjacent to the target site 10, a user may perform diagnostics of the tissue 12, 14 located therein with the combined device 150. For example, at step 204, the diagnostic sensing devices 156 of the medical instrument 140 may be activated to initiate sensing of the target site 10 in response to the processor 104 of the diagnostic computing device 102 executing the diagnostic sensing logic 107. In this instance, the diagnostic sensing devices 156 may detect, measure, record, and transmit sensor readings from the target site 10 to the diagnostic computing device 102 for analysis and diagnosis. With the plurality of diagnostic sensing devices 156 disposed along an exterior surface of the expandable body 152, such as, for example, about an annular array on the combined device 150, the medical instrument 140 is configured to measure data from a plurality of regions and/or surfaces surrounding the target site 10.

At step 206 and referring to FIG. 4, with the sensor data received from the combined device 150 and stored within the memory 106, the processor 104 of the diagnostic computing device 102 may analyze the sensor data to determine a presence and location of any desirable tissue 12 and/or undesirable tissue 14 within the target site 10. In instances where the sensor readings obtained by the diagnostic sensing devices 156 are determined to not be indicative of an existence of undesirable tissue 14 in the target site 10, a user of the medical system 100 may move the medical device 110 and/or the medical instrument 140 to another target treatment site 10 at step 208. In the example, the processor 104 of the diagnostic computing device 102 may repeat steps 204 and 206 of the method 200 described above to determine whether the new target treatment site 10 includes any undesirable tissue 14.

Alternatively, in instances where the sensor readings obtained by the diagnostic sensing devices 156 are determined to be indicative of an existence of undesirable tissue 14 in the target site 10, the processor 104 of the diagnostic computing device 102 may execute the therapy process logic 108 to initiate delivery of energy (e.g., RF current) to the undesirable tissue 14 via the combined device 150.

At step 210 and referring to FIG. 3C, the processor 104 of the diagnostic computing device 102 transmits a signal to the generator 101 and causes the generator 101 to supply the combined device 150 with electrical energy (e.g., RF current) for delivery to the target site 10 by the plurality of energy delivery devices 158. In some examples, the processor 104 may only activate and/or supply a predetermined subset of the plurality of energy delivery devices 158 with electrical energy at least partially dependent on a respective position of the energy delivery devices 158 on the expandable body 152 relative to a location of the undesirable tissue 14. In other words, the therapy process logic 108, when executed by the processor 104 of the diagnostic computing device 102, may identify one or more energy delivery devices 158 of the plurality of energy delivery devices 158 that are positioned adjacent to and/or facing toward the undesirable tissue 14 relative to the target site 10. In this instance, an energy current A may only be applied to the undesirable tissue 14 and not applied to the desirable tissue 12 within the target site 10.

The diagnostic computing device 102 of the medical system 100 may be operable to determine the one or more subsets of the energy delivery devices 158 from the plurality of energy delivery devices 158 to supply electrical energy based on the diagnostic sensor data provided by the diagnostic sensing devices 156. For example, with the array of diagnostic sensing devices 156 alternating with the array of energy delivery devices 158 on an exterior surface of the expandable body 152, the diagnostic sensing logic 107, when executed by the processor 104, may identify the one or more subsets of the diagnostic sensing devices 156 from the plurality of diagnostic sensing devices 156 that measured characteristics indicative of the presence of undesirable tissue 14 in the target site 10. Accordingly, the therapy progress logic 108 of the diagnostic computing device 102, when executed by the processor 104, may supply electrical energy to the one or more energy delivery devices 158 that are positioned relatively adjacent to the one or more subsets of the diagnostic sensing devices 156 that detected and/or measured the undesirable tissue 14.

At step 212 and referring to FIG. 4, in some examples, the therapy progress logic 108 may cause the processor 104 to periodically evaluate a progress of the therapy treatment of the target site 10 after a predefined therapy cycle has lapsed. In the example, the predefined therapy cycle may be stored in the memory 106 of the diagnostic computing device 102 and may be adjustable by user input. In other examples, the predefined therapy cycle may be automatically adjusted by the processor 104 of the diagnostic computing device 102 based on various factors, including, but not limited to, a quantity of the undesirable tissue 14 measured by the plurality of diagnostic sensing devices 156. It should be understood that the predefined therapy cycle may include various suitable durations (e.g., seconds, minutes, etc.).

In some examples, the predefined therapy cycle may determine a proportionate multiplexing relationship between applying energy therapy and sensing parameters of the tissue. For example, the therapy progress logic 108 may determine a predefined therapy cycle of about 50% therapy treatment and about 50% sensing within a predetermined time threshold (e.g., a blend duty cycle), or about 6% therapy treatment and about 94% sensing within a predetermined time threshold (e.g., a coagulate cycle). It should be appreciated that the predefined therapy cycles shown and described herein are merely illustrative and, in some embodiments, may include other percentages of therapy treatment and sensing, and may include a range of values +\−10% of the stated values. It should be understood that the predefined duty cycle may be determined by various other suitable measures without departing from a scope of this disclosure. For example, the predefined therapy cycles determined by the therapy progress logic 108 may further identify a degree of power to be applied to the undesirable tissue 14, a pulsation frequency for applying energy within a therapy treatment cycle, and/or the like.

In response to determining that the predefined therapy cycle has not lapsed at step 212, the processor 104 of the diagnostic computing device 102 continues to supply electrical energy to the energy delivery devices 158 from the generator 101 at step 210. In this instance, the energy delivery devices 158 of the combined device 150 continue to deliver the energy current A to the undesirable tissue 14. Alternatively, in response to determining that the predefined therapy cycle has lapsed at step 212, the processor 104 of the diagnostic computing device 102, when executing the therapy progress logic 108, automatically ceases supplying the energy delivery devices 158 with electrical energy from the generator 101 at step 214. In this instance, delivery of the energy current A from the energy delivery devices 158 to the undesirable tissue 14 is ceased and the method 200 returns to step 204.

In this instance, the diagnostic sensing devices 156 of the combined device 150 may be reactivated to perform a diagnostic evaluation of a current state and/or condition of the target site 10, similar to that described above with respect to step 204. At step 206, with the sensor data received from the combined device 150 and stored within the memory 106, the processor 104 may analyze the sensor data to determine a continued presence of the undesirable tissue 14. In instances where the sensor readings obtained by the diagnostic sensing devices 156 are determined to not be indicative of an existence of the undesirable tissue 14, a user of the medical system 100 may move the medical device 110 and/or the medical instrument 140 to another target treatment site 10 at step 208.

In other words, the medical device 110 and/or the medical instrument 140 may be moved at step 208 when the measured electrical parameters are not indicative of undesirable tissue at the target site 10. In some examples, the method 200 at step 206 may include determining whether the measured electrical parameters at step 204 are outside of a predefined range, and/or are above or below a predefined threshold, that is indicative of undesirable tissue.

In the example, the processor 104 of the diagnostic computing device 102 may repeat steps 204 and 206 of the method 200 described above to determine whether the new target treatment site 10 includes any undesirable tissue 14. Alternatively, in instances where the sensor readings obtained by the diagnostic sensing devices 156 are determined to be indicative of a continued presence of the undesirable tissue 14 at the target site 10, the processor 104 may execute the therapy process logic 108 to continue delivery of energy (e.g., RF current) to the undesirable tissue 14 at step 210. It should be understood that the therapy progress logic 108 may cause the processor 104 to continue repeating steps 204, 206, 210, 212, and 214 until a predetermined portion of the undesirable tissue 14 is removed from the target site 10 (e.g., partially, substantially, entirely, etc.).

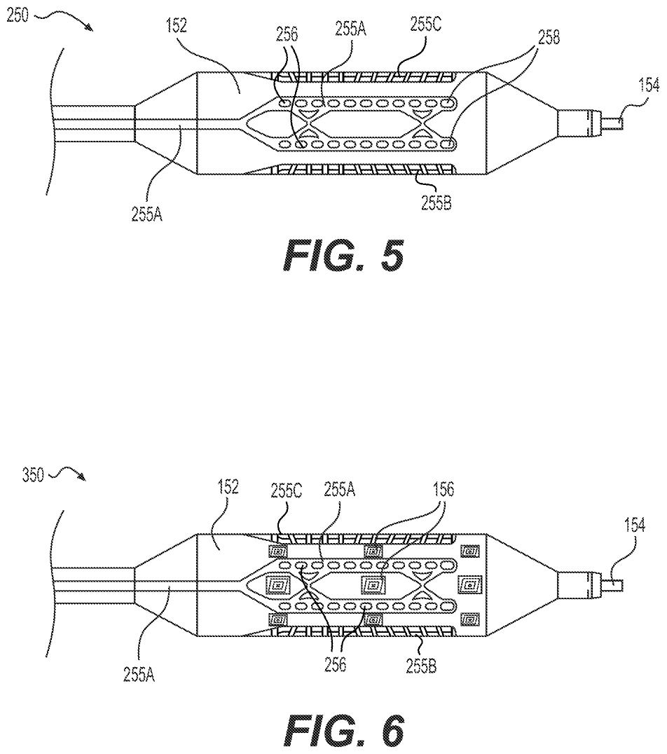

Referring now to FIG. 5, another exemplary combined device 250 is depicted in accordance with an example of this disclosure. Except as otherwise described below, the combined device 250 may be substantially similar to the combined device 150 described above such that like reference numerals are used to identify like components. Accordingly, it should be understood that the combined device 250 may be configured and operable like the combined device 150 except for the differences explicitly noted herein. Further, it should be understood that the combined device 250 may be readily incorporated into the medical instrument 140 described above such that an example of the medical instrument 140 that is equipped with the combined device 250 may be configured and operable similar to the medical instrument 140 described above.

The combined device 250 includes one or more electrode arrays 255 disposed about an exterior surface of the expandable body 152. Each of the one or more electrode arrays 255 (each denoted by numerals 255A, 255B, and 255C) may extend from a proximal end of the expandable body 152 to a distal end of the expandable body 152. The electrode arrays 255 may extend around a circumference of the combined device 250. Each of the one or more electrode arrays 255 may include one or more apertures 256 disposed thereon. In the example, the electrode array 255A includes a plurality apertures 256 extending along the electrode array 255A from a proximal end of the expandable body 152 to a distal end of the expandable body 152. In some examples, a geometry of the resonator and ground plane may be determinative of a position, size, and/or shape of each of the plurality of apertures 256 along the electrode array 255A, while in other examples a distance between an adjacent pair of energy delivery devices 258 in the electrode array 255 may be determinative of a position, size and/or shape of the apertures 256. The aperture 256 may be configured and operable to constrain a size of the spiral resonator (e.g., inductor) in the electrode array 255. In some examples, the aperture 256 may be further configured and operable to control a resolution of the target site (e.g., the undesirable tissue 14) by the one or more electrode arrays 255.

Still referring to FIG. 5, the one or more energy delivery devices 258 may be integrated into the one or more electrode arrays 255. In the example, the combined device 250 includes a pair of energy delivery devices 258 positioned at a distal end of each of the one or more electrode arrays 255. In this instance, the pair of energy delivery devices 258 may form a portion of the electrode arrays 255 or, in other instances, may be a separate component from the electrode arrays 255. In other examples, the energy delivery devices 258 may be positioned along various other portions of the electrode arrays 255.

The pair of energy delivery devices 258 may include electrodes, such as, for example, bipolar electrodes that are configured and operable similar to the energy delivery devices 158 and the diagnostic sensing devices 156 shown and described above. Accordingly, it should be appreciated that the pair of energy delivery devices 258 of the combined device 250 may be configured to detect and/or measure electrical parameters of a biological material at a target treatment site (e.g., tissue) and to deliver energy to a biological material at a target treatment site. In other words, the pair of energy delivery devices 258 may be operable to perform the functions and operations of the diagnostic sensing device 156 and the energy delivery device 158 described above.

Referring now to FIG. 6, another exemplary combined device 350 is depicted in accordance with an example of this disclosure. Except as otherwise described below, the combined device 350 may be substantially similar to the combined device 150, 250 described above such that like reference numerals are used to identify like components. Accordingly, it should be understood that the combined device 350 may be configured and operable like the combined device 150, 250 except for the differences explicitly noted herein. Further, it should be understood that the combined device 350 may be readily incorporated into the medical instrument 140 described above such that an example of the medical instrument 140 that is equipped with the combined device 350 may be configured and operable similar to the medical instrument 140 described above.

The combined device 350 includes one or more electrode arrays 255 and one or more diagnostic sensing devices 156 disposed about an exterior surface of the expandable body 152. The one or more electrode arrays 255 may be arranged and configured as described in the combined device 250 of FIG. 5. The one or more diagnostic sensing devices 156 may be disposed about the one or more electrode arrays 255 in a predefined pattern. In the example, the combined device 350 includes a plurality of diagnostic sensing devices 156 positioned about the one or more electrode arrays 255 at a plurality of locations along the expandable body 152. For example, the combined device 350 may include about three diagnostic sensing devices 156 positioned between adjacent electrode arrays 255, such as along the expandable body 152 between electrode arrays 255A, 255B and between electrode arrays 255A, 255C. Further, the combined device 350 may include about three diagnostic sensing devices 156 positioned along the expandable body 152 within intermediate regions of the electrode array 255A.

Each of the aforementioned systems, devices, assemblies, and methods may be used to detect, sense, measure, and treat a location of a target site. By providing a medical system including a diagnostic device and a therapy device, a user may accurately identify at-risk tissue and/or material within a subject's body using diagnostic sensing and ablation progress logic in a diagnostic computing device during a procedure, allowing a user to reduce overall procedure time, increase efficiency of procedures, and avoid unnecessary harm to a subject's body caused by misdiagnosis of tissue health at a target treatment site.

It will be apparent to those skilled in the art that various modifications and variations may be made in the disclosed devices and methods without departing from the scope of the disclosure. It should be appreciated that the disclosed devices may include various suitable computer systems and/or computing units incorporating a plurality of hardware components, such as, for example, a processor and non-transitory computer-readable medium, that allow the devices to perform one or more operations during a procedure in accordance with those described herein. Other aspects of the disclosure will be apparent to those skilled in the art from consideration of the specification and practice of the features disclosed herein. It is intended that the specification and examples be considered as exemplary only.

We claim:

1. A medical instrument, comprising:
a shaft; and
a device coupled to a distal end of the shaft, the device including:
(i) a sensor configured to measure an electrical parameter of tissue at a target site; and
(ii) an energy delivery device configured to deliver energy to the tissue at the target site based on the measured electrical parameter.

2. The medical instrument of claim 1, wherein the sensor and the energy delivery device are movable relative to the distal end of the shaft.

3. The medical instrument of claim 1, wherein the device includes an expandable body that is configured to expand laterally outward from a collapsed state to an expanded state.

4. The medical instrument of claim 3, wherein the sensor and the energy delivery device are positioned on the expandable body.

5. The medical instrument of claim 1, wherein the sensor includes one or more microwave antennas configured to sense dielectric permittivity of the tissue at the target site.

6. The medical instrument of claim 1, wherein the sensor includes one or more microwave or RF biosensors configured to sense parameters of the tissue at the target site.

7. The medical instrument of claim 1, wherein the sensor includes at least two electrodes configured to measure impedance between the at least two electrodes when positioned adjacent to the target site.

8. The medical instrument of claim 1, wherein the energy delivery device includes a microwave or RF electrode configured to transmit electric current to the target site and generate heat to ablate the tissue at the target site.

9. The medical instrument of claim 1, further comprising a processor and non-transitory computer readable medium storing instructions that, when executed by the processor, causes the processor to:
activate the energy delivery device when the electrical parameter measured by the sensor is indicative of undesirable tissue at the target site.

10. The medical instrument of claim 9, wherein the instructions stored in the non-transitory computer readable medium causes the processor to:
determine whether the electrical parameter measured by the sensor is indicative of undesirable tissue at the target site; and
transmit electrical energy from a generator to the energy delivery device in response to determining the electrical parameter is indicative of undesirable tissue.

11. The medical instrument of claim 10, wherein the instructions stored in the non-transitory computer readable medium cause the processor to:
periodically re-measure the electrical parameter at the target site with the sensor after delivering electrical energy from the energy deliver device to the tissue at the target site.

12. The medical instrument of claim 11, wherein the instructions stored in the non-transitory computer readable medium cause the processor to:
cease delivering electrical energy from the energy delivery device to the tissue at the target site when the electrical parameter measured by the sensor is not indicative of undesirable tissue at the target site.

13. The medical instrument of claim 11, wherein the instructions stored in the non-transitory computer readable medium cause the processor to:
cease transmission of electrical energy from the generator to the energy delivery device when the electrical parameter measured by the sensor is not indicative of undesirable tissue at the target site.

14. The medical instrument of claim 1, further comprising a plurality of sensors disposed along an exterior of the device in a first array, and a plurality of energy delivery devices disposed along the exterior of the device in a second array.

15. The medical instrument of claim 14, wherein the device is configured to locate the undesirable tissue relative to the exterior of the device based on a spatial distribution of the first array of the plurality of sensors.

16. A medical instrument comprising:
a flexible shaft having an articulable distal end; and
a device coupled to the articulable distal end of the shaft, the device comprising:
an expandable body;
a sensor array disposed along the expandable body and configured to measure an electrical parameter of tissue at a target site; and
an electrode array disposed along the expandable body and configured to heat the tissue at the target site;
wherein the expandable body is expandable to position the sensor array and the electrode array adjacent to the tissue at the target site.

17. The medical instrument of claim 16, wherein the sensor array includes a plurality of biosensors and the electrode array includes a plurality of electrodes; and
wherein the plurality of biosensors are disposed along the expandable body and alternate with the plurality of electrodes.

18. The medical instrument of claim 16, further comprising a processor and non-transitory computer readable medium storing instructions that, when executed by the processor, causes the processor to:
determine whether the electrical parameter measured by the sensor array is indicative of undesirable tissue at the target site; and
activate the electrode array when the electrical parameter measured by the sensor array is indicative of undesirable tissue at the target site.

19. The medical instrument of claim 18, wherein the instructions stored in the non-transitory computer readable medium causes the processor to:
periodically re-measure the electrical parameter at the target site with the sensor array after delivering electrical energy from the electrode array to the tissue at the target site; and
cease delivering electrical energy from the electrode array to the tissue at the target site when the electrical parameter measured by the sensor is not indicative of undesirable tissue at the target site.

20. A method of treating a target site with a medical device, the method comprising:
(a) measuring an electrical parameter of tissue at the target site with a sensor of the medical device;
(b) determining whether the measured electrical parameter is indicative of undesirable tissue;
(c) actuating an energy delivery device of the medical device if the electrical parameter is indicative of undesirable tissue, wherein actuation of the energy delivery device ablates the undesirable tissue; and
(d) repeating steps (a) through (c) until the electrical parameter measured at step (a) is determined to not be indicative of undesirable tissue at step (b).
